(19) United States
(12) Patent Application Publication
(10) Pub. No.: US 2008/0226654 A1
(43) Pub. Date: Sep. 18, 2008

(54) Uses of mammalian cytokine; related reagents

(75) Inventors: J. Fernando Bazan, Menlo Park, CA (US); Amy M. Beebe, Half Moon Bay, CA (US); Rene de Waal Malefyt, Sunnyvale, CA (US); Daniel M. Gorman, Palo Alto, CA (US); Peter Kirk, San Carlos, CA (US); Hirokazu Kurata, Palo Alto, CA (US); Donna Rennick, Los Altos, CA (US)

Correspondence Address: DNAX RESEARCH INC.;LEGAL DEPARTMENT, 901 CALIFORNIA AVENUE, PALO ALTO, CA, 94304, US

(73) Assignee: Schering Corporation, Kenilworth, NJ (US)

(21) Appl. No.: 11/437,391

(22) Filed: Jul. 13, 2006

Related U.S. Application Data
(63) Continuation of application No. 10/213,288, filed on Aug. 6, 2002, now abandoned.
(60) Provisional application No. 60/311,027, filed on Aug. 8, 2001.

(51) Int. Cl. A61K 39/395; C12N 5/00; A61P 19/08

(52) U.S. Cl. 424/158.1; 435/375

(57) ABSTRACT

Provided are methods of treatment for bone disorders related to bone deposition and resorption dysfunction. These bone disorders may be the result of inflammation, cancers, etc. Provided are methods of using agonists or antagonists of a cytokine molecule.

This filing is a continuation of co-pending U.S. patent application Ser. No. 10/213,288, which claims benefit of U.S. Provisional Patent Application No. 60/311,027, filed Aug. 8, 2001, each of which is incorporated herein by reference.

### FIELD OF THE INVENTION

The present invention relates generally to uses of mammalian cytokine-like molecules and related reagents. More specifically, the invention relates to identification of mammalian cytokine-like proteins and inhibitors thereof that affect medical conditions such as bone and inflammatory disorders.

### BACKGROUND OF THE INVENTION

The skeletal system, at first glance, appears to be a rigidly fixed and unchanging entity. However, this system is actually the result of a dynamic process involving a carefully regulated equilibrium between bone matrix deposition and resorption. These opposing actions are mediated through various bone associated cells including osteoblasts and osteoclasts, respectively.

Osteoblasts, the primary type of bone forming cell, are located on the surface of bone. These cells synthesize, transport and arrange many bone matrix proteins. Osteoblasts express several cell surface receptors including those for hormones (e.g., parathyroid hormone, Vitamin D, and estrogen), cytokines, and growth factors. (See, Cotrane, et al. (eds.) (1994) Robbins: Pathologic Basis of Disease, W.B. Saunders Company, Philadelphia, Pa.)

Osteoclasts are the cells responsible for bone resorption. These multinucleated granulocyte-monocyte lineage derived cells are also located on the surface of the bone. Osteoclasts are responsible for the release of a multitude of enzymes that act on disassembly of the matrix proteins into amino acids. The osteoclast released enzymes also activate certain growth factors and other enzymes, such as collagenase.

The opposing actions of osteoblasts and osteoclasts must be kept in balance to maintain skeletal integrity and calcium metabolism. The most common problem associated with dysregulation of this balance occurs when the rate of resorption exceeds the rate of deposition. This results in a loss of bone mass, e.g., as seen in osteoporosis, inflammatory conditions such as rheumatoid arthritis, and many cancers. (See, e.g., Rodan and Martin, (2000) Science 289:1508-1514.)

Some molecular mechanisms of osteoclast regulation have recently been discovered. One such molecule, RANKL, has been implicated in enhanced osteoclast function. (See, e.g., Suda et al. (1999) Endocr. Rev. 20:345-357.) RANKL, a TNF family member, is normally expressed on osteoblasts and activated T cells, binds to RANK which is expressed on osteoclasts and dendritic cells. (See, e.g., Wong, et al. (1999) J. Leuk. Biol. 65:715-724). When RANK is activated via binding to RANKL, a cascade of signaling molecules are activated resulting in the maturation of osteoclasts and enhancement of their bone resorption functions.

The balance of bone resorption and deposition will most likely involve a complex network of several regulatory molecules. As yet, many of these molecules and their functions have not been elucidated. The present invention satisfies this need.

### BRIEF DESCRIPTION OF THE DRAWING

FIG. 1 shows a sequence alignment between human HEMAE80 polypeptide (SEQ ID NO:2) and mouse HEMAE80 polypeptide (SEQ ID NO:4).

### SUMMARY OF THE INVENTION

The present invention is based, in part, upon the discovery of the role of a cytokine-like molecule, HEMAE80, in bone deposition and resorption.

The present invention provides methods of modulating bone resorption by contacting a cell with an agonist of HEMAE80; an antagonist of HEMAE80. HEMAE80 further comprises a polypeptide sequence of SEQ ID NO:2 or SEQ ID NO:4. In another embodiment, the antagonist of HEMAE80 is an antibody or binding fragment thereof. The antibody is a polyclonal antibody; or a monoclonal antibody.

The present invention also provides for a method of treating a bone resorption disorder comprising administering an antagonist of HEMAE80. In another embodiment, HEMAE80 is administered in combination with another therapeutic. In yet another embodiment, the antagonist is an antibody which specifically binds HEMAE80 and prevents loss of bone mineral density. The disorders embodied are: osteoporosis; osteopetrosis; Paget's disease; osteodystrophy; a result of a hemopoietic stem/progenitor cell (HSPC) hyperplasia; a result of an immune disorder; or a result of a cancer.

The present invention further embodies a method of treating a bone deposition disorder comprising administering an agonist of HEMAE80. The agonist inhibits bone deposition and is used to treat: excessive ossification of skeletal bone; ossification of cartilage; or Van Buchem's disease.

### DETAILED DESCRIPTION OF THE PREFERRED EMBODIMENTS

### I. General

The characteristic of the skeletal system is a dynamic entity whose health is predicated on a delicate equilibrium between bone deposition and bone resorption. Osteoblasts and osteoclasts are the key cell types responsible for this regulatory processes

The present invention resulted from studies on a secreted cytokine molecule, HEMAE80. HEMAE80 appears to be expressed by CD34+ hematopoietic stem/progenitor cells (HSPCs; see, e.g., Liu, et al. (2000) Genomics 65:283-292), cartilage, chondrocytes, endothelial cells, and osteoblasts. Quantitative PCR in human and mouse cells and tissues showed high levels of expression in mouse aorta and ears (skin/cartilage tissue). In the human, high expression was found in fetal spleen, fetal gall bladder, CD14+ ex vivo derived dendritic cells, and macrophages.

HSPCs reside in the bone marrow, in contact with bone marrow stromal cells. Stromal cells of the bone marrow are of the same lineage as osteoblasts and there is evidence for phenotype plasticity allowing interconversion of the stromal cells and osteoblasts (see, e.g., Krebsbach, et al. (1999) Crit. Rev. Oral Biol. Med. 10:165-181). Therefore, HSPCs may secrete a factor that affects osteoblast development. This is evidenced by the fact that hyperplasia of HSPCs can lead to osteoporosis (see, e.g., Ascenzi (1976) The Biochemistry and Physiology of Bone, G. Bourne (ed.) Academic Press, New York, N.Y.; and Tunaci, et al. (1999) Eur. Radiol. 9:1804-1809). Further, during skeletal development, invasion of nascent bone by HSPCs is required to prevent excessive ossification (see, e.g., Tavassoli and Yoffey (eds.)(1983) Bone Marrow: Structure and Function, Academic Press, New York, N.Y.; Gotoh, et al. (1995) Calcif. Tissue Int. 56:246-251; and Gundle et al. (1995) Bone 16:597-601).

Chondrocytes are the main cell type of cartilage, and are also of the bone marrow/osteoblast lineage. In order to prevent ossification of cartilage tissue, osteoblast activity must be inhibited to prevent entry into cartilage tissue. In view of their shared lineage, chondrocytes are also a potential target of HEMAE80.

Adenovirus delivery of HEMAE80 protein to mice resulted in significantly less bone mineral density in treated vs. untreated animals. Neonatal mice treated with purified human HEMAE80 protein had lower bone mineral density than mice receiving control treatment. Administration of purified human HEMAE80 protein to adult mice led to a 10% reduction in bone mineral density compared to pre-treatment levels, and significantly reduced limb bone length compared to control-treated animals. Finally both adenoviral and protein delivery of HEMAE80 to SCID mice implanted with human bone resulted in differences in comparison with normal bone. In particular, HEMAE80 treated bone had reduced marrow cellularity, uniform acellular deposits, and cell adhering to bone spinacles. Taken together, HEMAE80 can be a target for modulation of bone resorption and deposition.

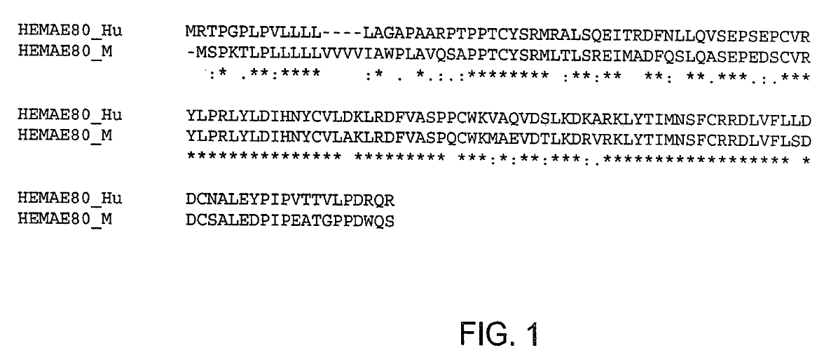

Structurally, human HEMAE80 polynucleotide (SEQ ID NO:1) encodes a polypeptide (SEQ ID NO:2), characteristic of the hematopoietic cytokine family having 4 α-helix bundle structures. Similarly, mouse HEMAE80 polynucleotide (SEQ ID NO:3) encodes a polypeptide (SEQ ID NO:4). FIG. 1 shows a sequence alignment between human HEMAE80 polypeptide (SEQ ID NO:2) and mouse HEMAE80 polypeptide (SEQ ID NO:4).

Secretion of HEMAE80 from CD34+ hematopoietic progenitor cells suggests that this molecule may have additional activities on the differentiation and proliferation of immune cells. HEMAE80 was mapped to human chromosome 4 in a region known for recurrent chromosomal translocations [t(4;14)(p16.3; q32)] in Multiple Myeloma. This suggests that HEMAE80 may be the target of this abnormality and may contribute to its tumorigenesis.

### II. Antagonists and Agonists

Blockage of the activities of HEMAE80 can be achieved by antagonists of the cytokine, e.g., antibodies to the ligand, antibodies to the receptor, etc. Interference with the ligand-receptor interaction has proven to be an effective strategy for the development of antagonists.

There are various means to antagonize the activity mediated by ligand. Two apparent means are to block the ligand with antibodies; a second is to block the receptor with antibodies. Various epitopes will exist on each which will block their interaction, e.g., causing steric hindrance blocking interaction. The correlation of ability to block signaling would not necessarily be expected to correlate with binding affinity to either ligand or receptors. Another means is to use a ligand mutein which retains receptor binding activity, but fails to induce receptor signaling. The mutein may be a competitive inhibitor of signaling ligand.

Alternatively, small molecule libraries may be screened for compounds which may block the interaction or signaling mediated by an identified ligand-receptor pairing.

The present invention provides for the use of an antibody or binding composition which specifically binds to a specified cytokine ligand, preferably mammalian, e.g., primate, human, cat, dog, rat, or mouse. Antibodies can be raised to various cytokine proteins, including individual, polymorphic, allelic, strain, or species variants, and fragments thereof, both in their naturally occurring (full-length) forms or in their recombinant forms. Additionally, antibodies can be raised to receptor proteins in both their native (or active) forms or in their inactive, e.g., denatured, forms. Anti-idiotypic antibodies may also be used.

A number of immunogens may be selected to produce antibodies specifically reactive with ligand or receptor proteins. Recombinant protein is a preferred immunogen for the production of monoclonal or polyclonal antibodies. Naturally occurring protein, from appropriate sources, e.g., primate, rodent, etc., may also be used either in pure or impure form. Synthetic peptides, made using the appropriate protein sequences, may also be used as an immunogen for the production of antibodies. Recombinant protein can be expressed and purified in eukaryotic or prokaryotic cells as described, e.g., in Coligan, et al. (eds. 1995 and periodic supplements) Current Protocols in Protein Science John Wiley & Sons, New York, N.Y.; and Ausubel, et al (eds. 1987 and periodic supplements) Current Protocols in Molecular Biology, Greene/Wiley, New York, N.Y. Naturally folded or denatured material can be used, as appropriate, for producing antibodies. Either monoclonal or polyclonal antibodies may be generated, e.g., for subsequent use in immunoassays to measure the protein, or for immunopurification methods.

Methods of producing polyclonal antibodies are well known to those of skill in the art. Typically, an immunogen, preferably a purified protein, is mixed with an adjuvant and animals are immunized with the mixture. The animal's immune response to the immunogen preparation is monitored by taking test bleeds and determining the titer of reactivity to the protein of interest. For example, when appropriately high titers of antibody to the immunogen are obtained, usually after repeated immunizations, blood is collected from the animal and antisera are prepared. Further fractionation of the antisera to enrich for antibodies reactive to the protein can be performed if desired. See, e.g., Harlow and Lane; or Coligan. Immunization can also be performed through other methods, e.g., DNA vector immunization. See, e.g., Wang, et al. (1997) Virology 228:278-284.

Monoclonal antibodies may be obtained by various techniques familiar to researchers skilled in the art. Typically, spleen cells from an animal immunized with a desired antigen are immortalized, commonly by fusion with a myeloma cell. See, Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) Cell and Tissue Culture: Laboratory Procedures, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies or binding compositions, including binding fragments and single chain versions, against predetermined fragments of ligand or receptor proteins can be raised by immunization of animals with conjugates of the fragments with carrier proteins. Monoclonal antibodies are prepared from cells secreting the desired antibody. These antibodies can be screened for binding to normal or defective protein. These monoclonal antibodies will usually bind with at least a K_{D} of about 1 mM, more usually at least about 300 μM, typically at least about 10 μM, more typically at least about 30 μM, preferably at least about 10 μM, and more preferably at least about 3 μM or better.

In some instances, it is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) Basic and Clinical Immunology (4th ed.) Lange Medical Publications, Los Altos, Calif., and references cited therein; Harlow and Lane (1988) Antibodies: A Laboratory Manual CSH Press; Goding (1986) Monoclonal Antibodies: Principles and Practice (2d ed.) Academic Press, New York, N.Y.; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve selection of libraries of antibodies in phage or similar vectors. See, e.g., Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see, Cabilly, U.S. Pat. No. 4,816,567; and Queen, et al. (1989) Proc. Nat'l Acad. Sci. USA 86:10029-10033; or made in transgenic mice, see Mendez, et al. (1997) Nature Genetics 15:146-156; also see Abgenix and Medarex technologies.

Antibodies are merely one form of specific binding compositions. Other binding compositions, which will often have similar uses, include molecules that bind with specificity to ligand or receptor, e.g., in a binding partner-binding partner fashion, an antibody-antigen interaction, or in a natural physiologically relevant protein-protein interaction, either covalent or non-covalent, e.g., proteins which specifically associate with desired protein. The molecule may be a polymer, or chemical reagent. A functional analog may be a protein with structural modifications, or may be a structurally unrelated molecule, e.g., which has a molecular shape which interacts with the appropriate binding determinants. Antibody binding compounds, including binding fragments, of this invention can have significant diagnostic or therapeutic value. They can be useful as non-neutralizing binding compounds and can be coupled to toxins or radionuclides so that when the binding compound binds to the antigen, a cell expressing it, e.g., on its surface, is killed. Further, these binding compounds can be conjugated to drugs or other therapeutic agents, either directly or indirectly by means of a linker, and may effect drug targeting.

Agonists include the cytokine protein identified (see, e.g., SEQ ID NO:2 and 4). Proteins of those sequences, or variants thereof, will be used to induce receptor signaling.

### III. Diagnostic Uses; Therapeutic Compositions, Methods

The invention provides means to address various bone, immune, or cancer related disorders. The etiology and pathogenesis are often not well understood, but they cause significant discomfort or morbidity in patients. As noted above, administration of the purified or adenovirus produced protein results in loss of bone density in various animal models. Collectively these studies suggest that agonizing or antagonizing this cytokine or its receptor, with the appropriate entity may offer a therapeutic modality in bone, immune, or cancer related disorders.

Diagnostic methods include such aspects as prediction of prognosis; definition of subsets of patients who will either respond or not respond to a particular therapeutic course; diagnosis of bone or immune related disorders or subtypes of these disorders; or assessing response to therapy. Antagonists or agonists to HEMAE80 activity can implicated in a manner suggesting significant therapeutic effects, e.g., to decrease or prevent occurrence of symptoms. The antagonists and/or agonists of the present invention can be administered alone or in combination with another inhibitor or agonist of the same or accompanying pathway; or other compounds used for the treatment of symptoms, e.g., antagonists, or steroids such as glucocorticoids.

Certain antagonists or agonists may be useful to slow down the process of bone density loss or ossification. Prevention of bone loss in disorders including but not limited to, osteoporosis, osteodystrophy, osteopetrosis, bone loss as a result of immune or cancer related disorders, or Paget's disease, would be advantageous. Enhanced bone resorption would be similarly be advantageous in disorders including, but not limited to, skeletal ossification, cartilage ossification, and Van Buchem's disease.

This may be effected by either direct administration of the agonist or antagonist, or perhaps using a gene therapy strategy. Antagonism may be effected, e.g., by antisense treatment, antibodies, or other suppression of HEMAE80 effects.

To prepare pharmaceutical or sterile compositions including the antibody, binding composition thereof, cytokine agonist, or small molecule antagonist, the entity is admixed with a pharmaceutically acceptable carrier or excipient which is preferably inert. Preparation of such pharmaceutical compositions is known in the art, see, e.g., Remington's Pharmaceutical Sciences and U.S. Pharmacopeia: National Formulary, Mack Publishing Company, Easton, Pa. (1984).

Antibodies, binding compositions, or cytokines are normally administered parentally, preferably intravenously. Since such proteins or peptides may be immunogenic they are preferably administered slowly, either by a conventional IV administration set or from a subcutaneous depot, e.g. as taught by Tomasi, et al, U.S. Pat. No. 4,732,863. Means to minimize immunological reactions may be applied. Small molecule entities may be orally active.

When administered parenterally the biologics will be formulated in a unit dosage injectable form (solution, suspension, emulsion) in association with a pharmaceutically acceptable parenteral vehicle. Such vehicles are typically inherently nontoxic and nontherapeutic. The therapeutic may be administered in aqueous vehicles such as water, saline, or buffered vehicles with or without various additives and/or diluting agents. Alternatively, a suspension, such as a zinc suspension, can be prepared to include the peptide. Such a suspension can be useful for subcutaneous (SQ) or intramuscular (IM) injection. The proportion of biologic and additive can be varied over a broad range so long as both are present in effective amounts. The antibody is preferably formulated in purified form substantially free of aggregates, other proteins, endotoxins, and the like, at concentrations of about 5 to 30 mg/ml, preferably 10 to 20 mg/ml. Preferably, the endotoxin levels are less than 2.5 EU/ml. See, e.g., Avis, et al. (eds.) (1993) Pharmaceutical Dosage Forms: Parenteral Medications 2d ed., Dekker, NY; Lieberman, et al. (eds. 1990) Pharmaceutical Dosage Forms: Tablets 2d ed., Dekker, NY; Lieberman, et al. (eds. 1990) Pharmaceutical Dosage Forms: Disperse Systems Dekker, NY; Fodor, et al. (1991) Science 251:767-773, Coligan (ed.) Current Protocols in Immunology; Hood, et al. Immunology Benjamin/Cummings; Paul (ed.) Fundamental Immunology; Academic Press; Parce, et al. (1989) Science 246:243-247; Owicki, et al. (1990) Proc. Nat'l Acad. Sci. USA 87:4007-4011; and Blundell and Johnson (1976) Protein Crystallography, Academic Press, New York.

Selecting an administration regimen for a therapeutic depends on several factors, including the serum or tissue turnover rate of the entity, the level of symptoms, the immunogenicity of the entity, and the accessibility of the target cells, timing of administration, etc. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate antibody doses is found in, e.g. Bach et al., chapter 22, in Ferrone, et al. (eds. 1985) Handbook of Monoclonal Antibodies Noges Publications, Park Ridge, N.J.; and Haber, et al. (eds.) (1977) Antibodies in Human Diagnosis and Therapy, Raven Press, New York, N.Y. (Russell, pgs. 303-357, and Smith, et al., pgs. 365-389). Alternatively, doses of cytokine or small molecules are determined using standard methodologies.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

The total weekly dose ranges for antibodies or fragments thereof, which specifically bind to ligand or receptor range generally from about 10 μg, more generally from about 100 μg, typically from about 500 μg, more typically from about 1000 μg, preferably from about 5 mg, and more preferably from about 10 mg per kilogram body weight. Generally the range will be less than 100 mg, preferably less than about 50 mg, and more preferably less than about 25 mg per kilogram body weight. Agonist or small molecule therapeutics may be used at similar molarities.

The weekly dose ranges for antagonists of cytokine receptor mediated signaling, e.g., antibody or binding fragments, range from about 1 μg, preferably at least about 5 μg, and more preferably at least about 10 μg per kilogram of body weight. Generally, the range will be less than about 1000 μg, preferably less than about 500 μg, and more preferably less than about 100 μg per kilogram of body weight. Dosages are on a schedule which effects the desired treatment and can be periodic over shorter or longer term. In general, ranges will be from at least about 10 μg to about 50 mg, preferably about 100 μg to about 10 mg per kilogram body weight. Cytokine agonists or small molecule therapeutics will typically be used at similar molar amounts, but because they likely have smaller molecular weights, will have lesser weight doses.

The present invention also provides for administration of biologics in combination with known therapies, e.g., steroids, particularly glucocorticoids, which alleviate the symptoms, e.g., associated with inflammation, or antibiotics or anti-infectives. Daily dosages for glucocorticoids will range from at least about 1 mg, generally at least about 2 mg, and preferably at least about 5 mg per day. Generally, the dosage will be less than about 100 mg, typically less than about 50 mg, preferably less than about 20 mg, and more preferably at least about 10 mg per day. In general, the ranges will be from at least about 1 mg to about 100 mg, preferably from about 2 mg to 50 mg per day. Suitable dose combinations with antibiotics, anti-infectives, or anti-inflammatories are also known.

The phrase “effective amount” means an amount sufficient to ameliorate a symptom or sign of the medical condition. Typical mammalian hosts will include mice, rats, cats, dogs, and primates, including humans. An effective amount for a particular patient may vary depending on factors such as the condition being treated, the overall health of the patient, the method route and dose of administration and the severity of side affects. When in combination, an effective amount is in ratio to a combination of components and the effect is not limited to individual components alone

An effective amount of therapeutic will decrease the symptoms typically by at least about 10%; usually by at least about 20%; preferably at least about 30%; or more preferably at least about 50%. The present invention provides reagents which will find use in therapeutic applications as described elsewhere herein, e.g., in the general description for treating disorders associated with the indications described above. Berkow (ed.) The Merck Manual of Diagnosis and Therapy, Merck & Co., Rahway, N.J.; Thorn, et al. Harrison's Principles of Internal Medicine, McGraw-Hill, NY; Gilman, et al. (eds.) (1990) Goodman and Gilman's: The Pharmacological Bases of Therapeutics, 8th Ed., Pergamon Press; Remington's Pharmaceutical Sciences, 17th ed. (1990), Mack Publishing Co., Easton, Pa.; Langer (1990) Science 249:1527-1533; Merck Index, Merck & Co., Rahway, N.J.; and Physician's Desk Reference (PDR); Cotran, et al. (eds), supra; and Dale and Federman (eds.) (2000) Scientific American Medicine, Healtheon/WebMD, New York, N.Y.

The broad scope of this invention is best understood with reference to the following examples, which are not intended to limit the inventions to the specific embodiments.

### EXAMPLES

### I. General Methods

Some of the standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Greene/Wiley, New York. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Meth. Enzymol., vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) QIAexpress: The High Level Expression & Protein Purification System QIAGEN, Inc., Chatsworth, Calif.

Computer sequence analysis is performed, e.g., using available software programs, including those from the GCG (U. Wisconsin) and GenBank sources. Public sequence databases were also used, e.g., from GenBank and others.

### II. Identification of HEMAE80

The human HEMAE80 clone was identified in the HGS (Rockville, Md.) database as a candidate secreted protein using the PSORT and SignalP leader prediction algorithms. HGS clone HEMAE80 in PC5 mammalian expression vector was prepared and sequence verified. 293T-cells were transiently transfected and newly synthesized proteins were metabolically labeled using [³⁵S] methionine. The transfection supernatant was analyzed on SDS PAGE. HEMAE80 transfected cells secrete a protein of 15 kD by IVR.

The mouse HEMAE80 clone was encoded by two public ESTs (BB568033, BF449650, dbEST), identified by BLAST using the coding region of human HEMAE80. The mouse ESTs were used to design oligonucleotide primers allowing amplification of the coding region of the gene by nested PCR, using Mouse 17-day embryo Marathon-ready cDNA (Clontech) as template. Incorporation of HindIII and NotI restriction sites into the cloning oligonucleotides allowed cloning into the adenoviral expression vectors.

### III. Production of HEMAE80 Protein

To generate an adenovirus transfer vector, the HEMAE80 clone was digested with NotI and XbaI and cloned into QBI AdCMV5-GFP-M#1 in the NotI and XbaI site. The vector and recombinant adenovirus production were as described in Hoek, R. et al, (2000) Science 290:1768-1771. Mammalian HEK293 cells were infected with this recombinant adenovirus and the supernatant was subjected to purification by cation exchange, anion exchange, followed by size exclusion chromatography. The protein product was characterized and quantified by SDS PAGE. Estimation of protein purity was ˜90%, based upon visualization of the SDS PAGE commassie stained gel. The endotoxin level in the preparation as measured by Limulus Amebocyte Lysate QCL-1000 (BioWhittaker) was 6.7 EU/ml.

### IV. Distribution of HEMAE80 Message

RNA was isolated from tissues or cultured cells either using RNA-easy mini kits (Qiagen, Valencia, Calif.) or RNAzol B (Tel-test Inc., Friendswood, Tex.), according to manufacturers instructions. 2 μg total RNA was reverse transcribed into cDNA using 500 ng Oligo (dT) 12-18 (Boehringer), 50 ng p(dN6) (Pharmacia, Peapack, N.J.), 0.5 mM dNTP mix (Boehringer), 5 mM DTT and 200 U Superscript II RNA'se H-RT (Gibco BRL) in a 20 μl reaction at 42° C. for 50 min. Alternatively, cDNAs from various libraries were used as templates for quantitative PCR.

Twenty to fifty nanograms of cDNA per reaction was used as template for quantitative PCR and analyzed for the expression of human cytokine, cytokine receptor, or transcription factor genes by the fluorogenic 5′-nuclease PCR assay [Holland, 1991 #28] using the GeneAmp 5700 or ABI Prism 7700 Sequence Detection Systems (Perkin-Elmer, Foster City, Calif.). The amplicons used for human HEMAE 80 covered bp 358-405, and mouse HEMAE 80 xx respectively (numbering starts at start codon), and were analyzed either using a FAM labeled probe or with the intercalating fluorochrome dye SYBR Green. Sequences were as follows:

hHEMAE80 forward:

(SEQ ID NO: 5)

ATGACTGCAATGCCTTGGAAT,
hHEMAE80 reverse:

(SEQ ID NO: 6)

TCCCTTAGCGCTGACGATCT,
and
hHEMAE80 probe:

(SEQ ID NO: 7)

6FAM-CCCAATCCCAGTGACTACGGTCCTGC-TAMRA.

Primers and probes detecting human cytokine and cytokine receptor expression were obtained as pre-developed assay reagents (PDAR's) (Perkin Elmer). PCR reactions were assembled using Taqman Universal PCR Master Mix or SYBR Green PCR master mix (ABI Foster City Calif.) according to the manufacturer's protocols to yield final concentrations of 1×PCR buffer, 200 μM dATP, dCTP, dGTP and 400 ∞M dUTP, 5.5 mM MgCl₂, 1.25 U AMPLITAQ Gold DNA polymerase, Passive Reference I, and 0.5 U AMP-ERASE Uracil-N-glycosylase. Forward and reverse primers were added at 900 nM each and FAM labelled probes at 250 nM. The expression of 18S rRNA was measured in a multiplex assay in each sample and served as internal control for amount of input cDNA.

Multiplex assays contained 18S rRNA forward primer, reverse primer and VIC labeled probe at 50 nM in the PCR reaction. Forward and reverse primers were used at 200 nM in (singleplex) SYBR Green assays. The thermal cycling conditions included 50° C. for 2 min and 95° C. for 10 min, followed by 40 cycles of amplification at 95° C. for 15 s, and 55° C. for 1 min for denaturing and anneal-extension, respectively. Absolute quantitation was based on comparison to standard curves obtained by 10-fold serial dilutions of plasmid DNA containing the gene of interest as insert and corrected for amount of cDNA as determined by expression of the internal control.

### V. Adenoviral Delivery of HEMAE80 to C57BL/6J and Neonatal Mice

Adenoviral delivery of HEMAE80 to 14 week old C57BL/6J mice was performed as follows. Fourteen week old female C57/BL/6 mice were injected intravenously with 5×10̂10 replication deficient adenovirus expressing mHEMEA80 (SEQ ID NO:4) or control adenovirus expressing GFP. Fourteen days later, tibias were removed and the proximal ends were analyzed for trabecular bone mineral density by peripheral quantitative computed tomography (pQCT). Treated bones had significantly less trabecular bone than the control animals (p=0.029) (n=3 treated and 3 control).

Neonatal mice were given 6.5 microgram of human HEMAE80 protein every other day i.p. They received 8 i.p. injections totaling 52 micrograms/mouse. The mice were taken down a day after the last injection of protein and the tibias placed in 70% ethanol followed by bone density measurements.

### VI. Treatment of Neonatal and Adult Mice with Recombinant HEMAE80 Protein

Neonatal mice received a course of 8 i.p. injections of 6.5 micrograms of recombinant human HEMAE80 protein (or PBS control) on alternate days, totaling 52 micrograms per mouse. The mice were taken down one day after the last injection of protein and the bone mineral density of tibias analyzed by PQCT. Bone mineral density (BMD) was significantly reduced in HEMAE80-treated mice compared to animals receiving control injections (p=0.011).

Age- sex- and weight-matched adult Swiss-Webster mice received a course of 10 i.p. injections of 6.5 micrograms of recombinant human HEMAE80 protein (or PBS control) on alternate days (n=8 treated, 8 control). The BMD of the left tibia of each mouse was analyzed by pQCT before and after the course of treatment. Length of left tibia was measured after treatment. The change in BMD was significantly greater in HEMAE80-treated animals than in control animals (p=0.0105), with treated animals showing a mean BMD reduction of 10%. Mean tibia length was significantly shorter in HEMAE80-treated mice than in control mice (p=0.0217). HEMAE80-treatment did not affect body mass.

### VII. HEMAE80 in SCID Mice

Rag2 x gc −/− 129sv mice lack T cells, B cells and NK cells are immuno-compromised and can be transplanted with human tissues without adverse effects to constitute scid-hu mice. Human organs that have been successfully transplanted include fetal liver, fetal bone, fetal thymus, PBMC and fetal skin. Since HEMAE80 was found to be abundantly expressed in libraries of tissues containing high amounts of cartilage, rag2 x gc −/− mice were transplanted with human fetal bone to test its biological effects.

Human fetal bone pieces (2 per mouse) were transplanted at each flank under the skin and allowed to establish for a period of four weeks. Subsequently, Hemae80 was administered to the mice either as purified protein or as adenovirus expressing the HEMAE80 cDNA. Adenovirus expressing GFP (green fluorescent protein) or human HEMAE80 protein was administered iv at 10¹⁰ cfu. At sixteen days after administration, mice were sacrificed, human bones and human bones collected. One bone of each mouse was decalcified and examined by histology. Cells were collected from the colateral bone and the expression of genes involved in hematopoiesis and bone metabolism were analyzed by quantitative “real time” PCR following preparation of cDNA. In addition, hind legs of the mice were dissected and analyzed for bone characteristics including trabecular bone mineral density of the proximal tibia's. Treatment of animals with adeno-HEMAE80 resulted in a trend towards bone loss.

Hematoxylin and Eosin (HE) staining of human bone slides from adenovirus-GFP treated mice showed normal bone morphology with bone marrow containing hematopoietic cells including granulocytes. In contrast, slides from HEMAE80 treated mice showed a strong reduction in the cellularity of the bone marrow and a greater presence of stroma with probably deposition of connective tissue. In addition, bone fragments were flanked by large connective tissue cells. These results indicate that HEMAE80 has profound effects on bone marrow composition and bone morphology.

Quantitative PCR analyses indicated a 24 fold increase in the levels of Collagen type 10A; a 2.2 fold increase in the levels of collagen 7B; a 4 fold increase in the level of RANKL; a 2.5 fold increase in the levels of IL-6; and a 3 fold decrease in the level of CD14 in the cDNA samples prepared from HEMAE80 treated mice. This was specific since the expression levels of other genes involved in bone remodeling including IL-8, Collagen 1 A2, Collagen 5 A2, Collagen 6 A3, Timp1, IL-7, RANK, IL-1, CD40, gamma-c, GM-CSF, IL-3, IL-15 and TGF-β were not affected by more than 2 fold.

HEMAE 80 purified protein was administered ip at concentrations of 10, 2, and 0.4 microgram per mouse per day for 7 days. Mice were sacrificed, human bones collected, and cells harvested 24 hrs following the last administration. The expression of genes involved in hematopoiesis and bone metabolism were analyzed by quantitative “real time” PCR. In addition, hind legs of mice were dissected and analyzed for bone characteristics including trabecular mineral bone density.

Bone density measurements showed a slight decrease in trabecular bone. Quantitative PCR analyses also indicated a greater than 2 fold increase RNAK, RANKL, IL-1, IL-6, CD40, IL-15, and GCSF.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

1. A method of modulating bone resorption comprising contacting a cell with:
a) an agonist of HEMAE80; or
b) an antagonist of HEMAE80.

2. The method of claim 1, wherein HEMAE80 comprises a polypeptide sequence of SEQ ID NO:2 or SEQ ID NO:4.

3. The method of claim 1, wherein the antagonist of HEMAE80 is an antibody or binding fragment thereof.

4. The method of claim 1, wherein the antibody is:
a) a polyclonal antibody; or
b) a monoclonal antibody;

5. A method of treating a bone resorption disorder, said method comprising administering an antagonist of HEMAE80.

6. The method of claim 5, wherein said administering is in combination with another therapeutic.

7. The method of claim 5, wherein the antagonist is an antibody which specifically binds HEMAE80 and prevents loss of bone mineral density.

8. The method of claim 5, wherein the bone resorption disorder is:
a) osteoporosis;
b) osteopetrosis;
c) Paget's disease;
d) osteodystrophy:
e) a result of a hemopoietic stem/progenitor cell (HSPC) hyperplasia;
f) a result of an immune disorder; or
g) a result of a cancer.

9. A method of treating a bone deposition disorder, said method comprising administering an agonist of HEMAE80.

10. The method of claim 9, wherein the agonist inhibits bone deposition.

11. The method of claim 9, wherein the bone deposition disorder is:
a) excessive ossification of skeletal bone;
b) ossification of cartilage; or
c) Van Buchem's disease.


SEQUENCE LISTING (7 sequences)

SEQ ID NO: 1 - DNA, 984 bp, Homo sapiens
  CDS (12)..(419)
cgcctggcaccatgaggacgcctgggcctctgcccgtgctgctgctgctcctggcgggagcccccgccgcgcggcccactcccccgacctgctactcccgcatgcgggccctgagccaggagatcacccgcgacttcaacctcctgcaggtctcggagccctcggagccatgtgtgagatacctgcccaggctgtacctggacatacacaattactgtgtgctggacaagctgcgggactttgtggcctcgcccccgtgttggaaagtggcccaggtagattccttgaaggacaaagcacggaagctgtacaccatcatgaactcgttctgcaggagagatttggtattcctgttggatgactgcaatgccttggaatacccaatcccagtgactacggtcctgccagatcgtcagcgctaagggaactgagaccagagaaagaacccaagagaactaaagttatgtcagctacccagacttaatgggccagagccatgaccctcacaggtcttgtgttagttgtatctgaaactgttatgtatctctctaccttctggaaaacagggctggtattcctacccaggaacctcctttgagcatagagttagcaaccatgcttctcattcccttgactcatgtcttgccaggatggttagatacacagcatgttgatttggtcactaaaaagaagaaaaggaccaacaagcttcacttttatgaacaactattttgagaacatgcacaatagtatgtttttattactggtttaatggagtaatggtacttttattctttcttgatagaaacctgcttacatttaaccaagcttctattatgcctttttctaacacagactttcttcactgtctttcatttaaaaagaaattaatgctcttaagatatatattttacgtagtgctgacaggacccactctttcattgaaaggtgatgaaaatcaaataaagaatctcttcacatgag

SEQ ID NO: 2 - protein, 136 aa, Homo sapiens
MRTPGPLPVLLLLLAGAPAARPTPPTCYSRMRALSQEITRDFNLLQVSEPSEPCVRYLPRLYLDIHNYCVLDKLRDFVASPPCWKVAQVDSLKDKARKLYTIMNSFCRRDLVFLLDDCNALEYPIPVTTVLPDRQR

SEQ ID NO: 3 - DNA, 613 bp, Mus musculus
  CDS (31)..(447)
gagctgacccagcagtaggcaccaggcaccatgtcaccaaaaacactacctctgttgctgctgctggtggtggtggtgatagcctggcctctggcagtacagtccgcgccccccacctgctactctcggatgctgaccctgagccgtgagatcatggcagacttccagagcctgcaggcttcagagcctgaggattcctgtgtgaggtacttgccccggctttacctggacatccataactactgtgtgctggccaagctgagagacttcgtggcttctcctcagtgctggaagatggccgaagtggacactctgaaggacagagtgcggaagctgtataccatcatgaactccttctgcaggcgggacttggtattcctctcagatgactgcagtgccttagaagacccaattcccgaggccacgggtcctccagactggcagagctaagcaggtggaccagaagaacaacccagaggtctgaagctgggccagttgtccagagttacaccccccacacacacacccaggtctacttttagtgccactgttagacctgccacatgtctctagcttctgaaacaccagtgagggtcctacctctgagcatgct

SEQ ID NO: 4 - protein, 139 aa, Mus musculus
MSPKTLPLLLLLVVVVIAWPLAVQSAPPTCYSRMLTLSREIMADFQSLQASEPEDSCVRYLPRLYLDIHNYCVLAKLRDFVASPQCWKMAEVDTLKDRVRKLYTIMNSFCRRDLVFLSDDCSALEDPIPEATGPPDWQS

SEQ ID NO: 5 - DNA, 21 bp, Homo sapiens
atgactgcaatgccttggaat

SEQ ID NO: 6 - DNA, 20 bp, Homo sapiens
tcccttagcgctgacgatct

SEQ ID NO: 7 - DNA, 26 bp, Homo sapiens
cccaatcccagtgactacggtcctgc